(19) United States
(12) Patent Application Publication
(10) Pub. No.: US 2014/0111774 A1
(43) Pub. Date: Apr. 24, 2014

(54) OPHTHALMOLOGIC APPARATUS, OPHTHALMOLOGIC CONTROL METHOD, AND RECORDING MEDIUM

(71) Applicant: CANON KABUSHIKI KAISHA, Tokyo (JP)

(72) Inventor: Isao Komine, Sagamihara-shi (JP)

(73) Assignee: CANON KABUSHIKI KAISHA, Tokyo (JP)

(21) Appl. No.: 14/061,126

(22) Filed: Oct. 23, 2013

(30) Foreign Application Priority Data
Oct. 24, 2012 (JP) 2012-234648

(51) Int. Cl. A61B 3/00

(52) U.S. Cl. CPC A61B 3/0008
USPC 351/221; 351/246

(57) ABSTRACT

There are provided an ophthalmologic apparatus, an ophthalmologic control method, and a recording medium capable of irradiating a subject's eye with an appropriate light amount. The ophthalmologic apparatus includes a measurement unit configured to measure a light amount emitted from a light source, and a control unit configured to control a light amount emitted from the light source based on relation information representing a relationship between a light amount emitted from the light source and a light amount irradiated on a test eye, and the light amount measured by the measurement unit.

### BACKGROUND OF THE INVENTION

1. Field of the Invention

The present invention relates to an ophthalmologic apparatus, an ophthalmologic control method, and a recording medium.

2. Description of the Related Art

Currently, with the various standards for medical devices being prepared, there is a need to realize an apparatus that uses a light amount that is safe for a subject's eye even for ophthalmologic devices that perform inspection, measurement, and processing. Further, it is also necessary to improve apparatus performance in order to perform more accurate diagnosis for various subjects. Consequently, there is a need to utilize a high-power laser light source, for example. Therefore, there is a need for development of an excellent interlocking mechanism to ensure safety.

Japanese Patent Application Laid-Open No. 2011-27715 discusses an optical coherence tomography (OCT) apparatus in which a shutter is opened and closed to block a light path based on a light amount of reference light (executes measurement if the light amount of the reference light is within a permissible range and does not execute measurement if the light amount of the reference light is not within the permissible range).

However, the configuration discussed in Japanese Patent Application Laid-Open No. 2011-27715 includes a splitting unit that splits light from a light source into a measurement light and a reference light, and only detects the light amount of the reference light that is not incident on a subject's eye. Consequently, it is unclear what kind of effect the light amount of the laser light irradiated on the subject's eye has on the fundus or cornea surface of the subject's eye.

### SUMMARY OF THE INVENTION

The present invention is directed to an ophthalmologic apparatus, an ophthalmologic control method, and a recording medium capable of irradiating a subject's eye with an appropriate light amount by specifically focusing on the irradiation light amount of a subject's eye.

According to an aspect of the present invention, an ophthalmologic apparatus includes a measurement unit configured to measure a light amount emitted from a light source, and a control unit configured to control a light amount emitted from the light source based on relation information representing a relationship between a light amount emitted from the light source and a light amount irradiated on a subject's eye, and the light amount measured by the measurement unit.

According to another aspect of the present invention, an ophthalmologic control method includes measuring with a measurement unit a light amount emitted from a light source, and controlling a light amount emitted from the light source based on relation information representing a relationship between a light amount emitted from the light source and a light amount irradiated on a subject's eye, and the light amount measured by the measurement unit.

According to yet another aspect of the present invention, a non-transitory computer-readable recording medium stores an ophthalmologic control program for causing a computer to execute the ophthalmologic control method.

Further features and aspects of the present invention will become apparent from the following detailed description of exemplary embodiments with reference to the attached drawings.

### BRIEF DESCRIPTION OF THE DRAWINGS

FIG. 1A illustrates an example of a configuration of an ophthalmologic apparatus according to a first exemplary embodiment, and FIG. 1B illustrates an example of the function blocks in the ophthalmologic apparatus according to the first exemplary embodiment.

FIG. 2A illustrates an example of an ophthalmologic apparatus according to a second exemplary embodiment, and FIG. 2B illustrates an example of the function blocks in the ophthalmologic apparatus according to the second exemplary embodiment.

FIG. 3 illustrates an example of a shutter according to the second exemplary embodiment.

FIG. 4A is a flowchart according to the first exemplary embodiment, and FIG. 4B is a flowchart according to the second exemplary embodiment.

FIG. 5 illustrates an example of an ophthalmologic apparatus according to a third exemplary embodiment.

FIG. 6 is an example of a flowchart according to the third exemplary embodiment.

FIGS. 7A, 7B, 7C, and 7D illustrate examples of a relationship between a laser output amount and a laser irradiation amount that is irradiated on a cornea or a fundus of a subject's eye.

FIG. 8 is a perspective view of an alignment prism diaphragm according to an exemplary embodiment of the present invention.

FIGS. 9A, 9B, and 9C are diagrams illustrating a state in which alignment in the front-back direction performed using an alignment prism diaphragm is correct, too far, and too close, respectively.

### DESCRIPTION OF THE EMBODIMENTS

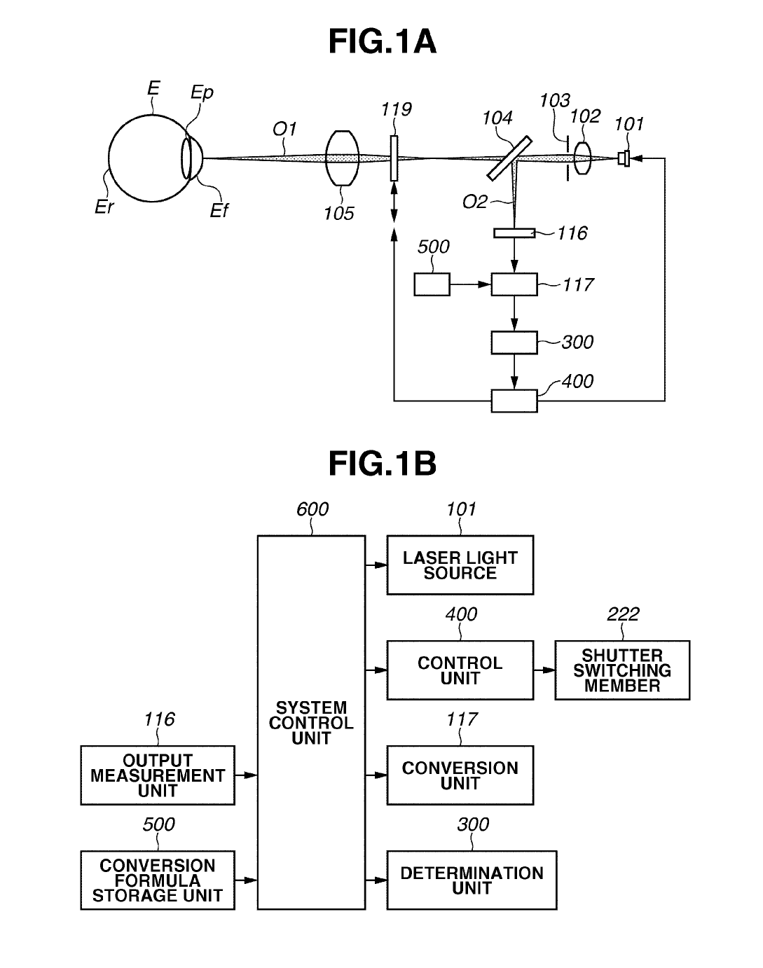

A first exemplary embodiment according to the present invention will now be described with reference to FIG. 1A. FIG. 1A illustrates a schematic example of a projection optical system of an ophthalmologic apparatus (e.g., an apparatus for performing laser processing on a predetermined site on a subject's eye) according to the present exemplary embodiment.

### (Overall Configuration)

The projection optical system includes a light source 101 for generating laser light. A super luminescent diode (SLD) or amplified spontaneous emission (ASE) can be used for the light source 101. Further, an ultrashort pulsed laser, such as a titanium-sapphire laser, may also be used for the light source 101. Thus, the light source 101 may be any light source, as long as it can generate laser light that includes low-coherence light.

The laser is a device that is capable of generating or amplifying electromagnetic radiation in the wavelength range of 180 nm to 1 mm, for example, by controlling dielectric emission. Therefore, the laser light is light with a wavelength range of 180 nm to 1 mm, for example, that is emitted by dielectric radiation. Considering the fact that an eye is to be measured, near-infrared light is suitable for this usage. Some other wavelengths may also be selected depending on the measurement site of the obsrvation target.

The projection optical system also includes lenses 102 and 105, and a diaphragm 103. These members configuring the projection optical system may be changed based on the type of the ophthalmologic apparatus. A light-splitting member 104, which is a half mirror or the like, splits the light in a light path between the light source 101 and a shutter 119 into a projection light beam light path 01 and a measuring laser output light path 02. The light irradiated from the light source 101 is guided to an output measurement unit 116 by the light-splitting member 104. The shutter 119 is controlled and driven by a control unit 400 to switch between blocking and passing through the projection light beam.

The shutter 119 may be a filter that controls the transmissivity of the light. In such a case, the shutter 119 may be configured so that it is capable of changing the amount of projected laser light. Further, the shutter 119 may perform the above-described switching by rotating or inserting/retracting an optical member such as a mirror. The output measurement unit 116 measures the laser output from the laser light source. This output measurement unit 116 may include a line sensor or a two-dimensional sensor as a charge-coupled device (CCD), or a power meter, as long as it can detect light.

The output measurement unit 116 is arranged at a conjugated position with the cornea or fundus of the subject's eye.

Further, a conversion unit 117 converts a laser output amount, such as the laser power of the laser light source, into a laser irradiation amount, such as a laser power, irradiated on the fundus or cornea surface of the subject's eye (i.e., functions as a determination unit for determining the amount of light incident on the subject's eye).

A determination unit 300 determines whether the irradiation light amount is within a permissible range based on an output from the conversion unit 117. Then, the control unit 400 performs the following control. Specifically, if it is determined by the determination unit 300 that the irradiation light amount is within the permissible range, the control unit 400 executes switching to a second state of the shutter 119 (shutter open in which the shutter is retracted from the light path). On the other hand, if it is determined by the determination unit 300 that the irradiation light amount is not within the permissible range, the control unit 400 maintains a first state (shutter closed state) of the shutter 119.

If it is determined by the determination unit 300 that the value calculated by the conversion unit 117 is not within the permissible range, the control unit 400 reduces the output of the light source 101. Then, the control unit 400 repeats the measurement by the output measurement unit 116, the conversion by the conversion unit 117, and the determination by the determination unit 300 until it is determined by the determination unit 300 that the irradiation light amount is within the permissible range. Thus, in the present exemplary embodiment, the output measurement unit 116, the conversion unit 117, the determination unit 300, and the control unit 400 are the constituent elements of a light source light amount adjustment apparatus.

### (Irradiation Light Amount Conversion Method Performed by the Light Source Light Amount Adjustment Apparatus)

The method according to the present exemplary embodiment for converting the output amount of the laser light source into a laser irradiation amount, such as a laser power, that is irradiated on the fundus or cornea surface of the subject's eye by the conversion unit 117 is as follows.

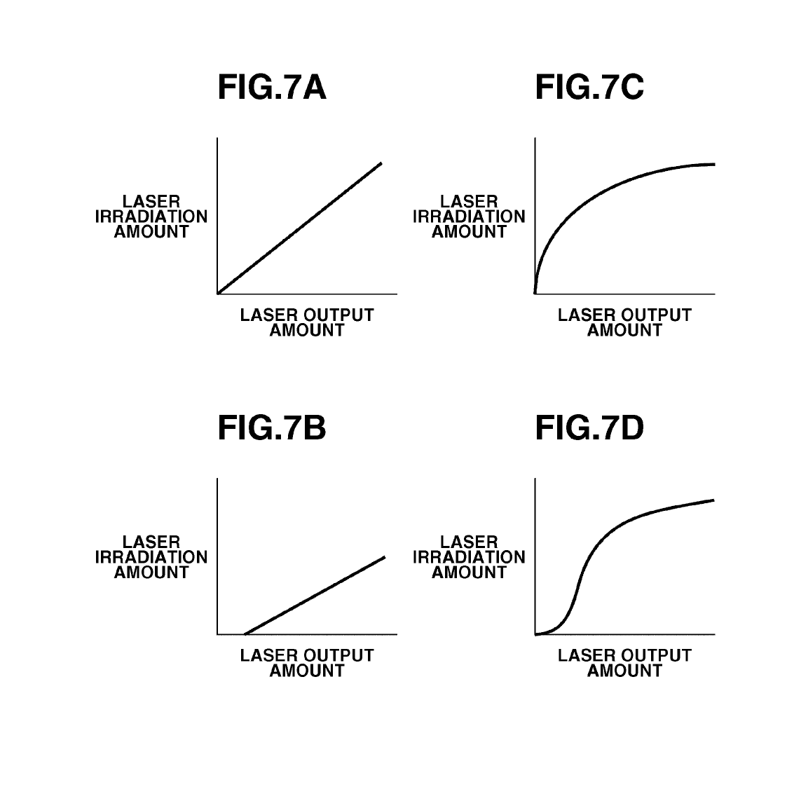

For the conversion, a conversion formula like that illustrated by the patterns in FIGS. 7A to 7D, for example, is used. More specifically, a conversion formula is used that is stored in a storage unit 117a as relation information representing the relationship between the output amount measured by the output measurement unit 116 and the laser irradiation amount irradiated on the cornea or fundus of the subject's eye via the optical system (members 102, 103, 104, and 105). This relation information is determined based on the rate of attenuation of the light amount on the light path from the light source to the subject's eye and the rate of attenuation in the light amount at the subject's eye.

When experimentally determining this conversion formula, a sensor such as a power meter is arranged at the cornea or the fundus position of a subject's eye E, and the shutter 119 is switched to the second state (shutter open). The measurement value at that time is taken as y. Further, if the measurement value of the output measurement sensor when the shutter 119 is switched to the first state (shutter closed) is taken as x, then a relational expression between y and x can be approximately derived. The conversion is performed by inputting that relational expression into the conversion unit 117 as a conversion table.

If the irradiation target is the fundus of the subject's eye, the conversion unit 117 outputs the light amount converted based on the rate of attenuation in the light amount resulting from the light beam passing through the optical system (members 102, 103, 104, and 105) and the rate of attenuation in the light amount resulting from the light beam passing through the inside of the subject's eye to the fundus of the subject's eye.

By using the above-described conversion formula, the actual laser irradiation amount on the subject's eye with respect to the laser output measurement value can be obtained based on the estimated rate of attenuation due to the membrane properties and the transmissivity of the optical members configuring the projection optical system, or based on experimentally-determined rate of attenuation data. Further, since the output measurement unit 116 is arranged at a conjugated position with the fundus of the subject's eye, and since the irradiated surface area at the laser output measurement position is correlated to the fundus image formation surface area, the effects on the subject's eye by the laser irradiation can be accurately known.

### (Laser Irradiation Determination)

The determination of laser irradiation on the subject's eye according to the present exemplary embodiment is performed as follows. With a state in which the shutter 119 is inserted into the light path 01 and the light path is blocked, the conversion unit 117 converts the received light amount into a laser irradiation amount to be irradiated on the fundus of the subject's eye. If the conversion result is within the permissible range (equal to or less than a predetermined value), the light path 01 is opened by the shutter 119. This predetermined value is, for example, the maximum value at which no harmful effects are given even if laser light is irradiated on the fundus or cornea surface of the subject's eye. However, the predetermined value is not limited to this, and for more safety, it may be a lower value than the maximum light amount at which no harmful effects are given on the subject's eye, for example.

If the conversion result is not within the permissible range (greater than the predetermined value), the control unit 400 functioning as a light amount adjustment unit reduces the laser light amount by controlling the current or the voltage of the laser light source 101, for example. The control unit 400 reduces the output of the light source 101, and repeats the measurement by the output measurement unit 116, the conversion by the conversion unit 117, and the determination by the determination unit 300 whether the conversion result is within the permissible range, until it is determined by the determination unit 300 that the conversion result is within the permissible range.

When the shutter 119 is in an open state (second state), if it is determined by the determination unit 300 that the conversion result is within the permissible range, the control unit 400 maintains the state in which the shutter 119 is open. On the other hand, if it is determined by the determination unit 300 that the conversion result is not within the permissible range, the control unit 400 performs the following control. Specifically, the control unit 400 switches the shutter 119 to the closed state (first state), reduces the output of the light source 101, and executes the measurement of the output by the output measurement unit 116, conversion by the conversion unit 117, and determination by the determination unit 300.

### (Flowchart of the Overall Apparatus)

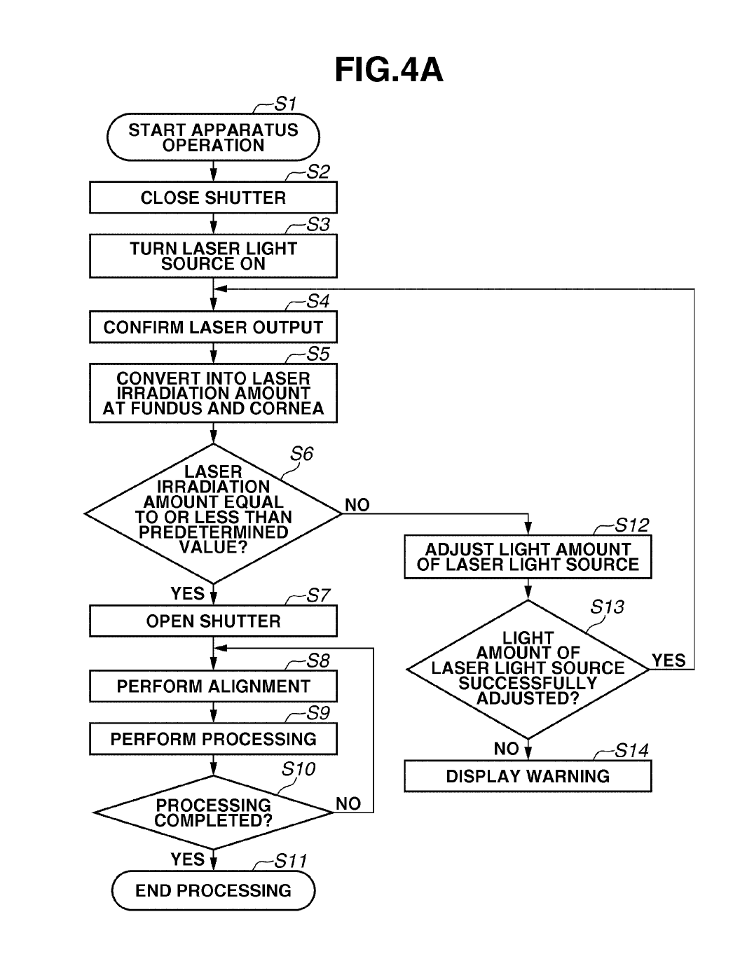

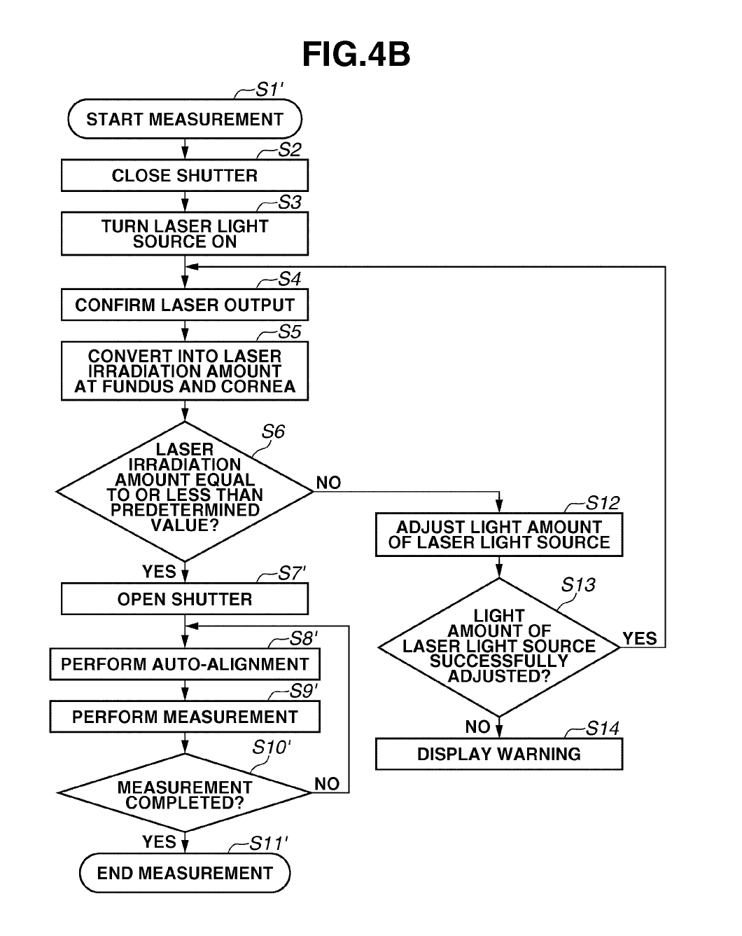

The above configuration will now be described with reference to the block diagram illustrated in FIG. 1B and the flowchart illustrated in FIG. 4A. A system control unit 600 illustrated in FIG. 1B performs overall control of the constituent elements, such as the laser light source 101, the control unit 400, the conversion unit 117, and the determination unit 300.

In step S1 of FIG. 4A, the apparatus starts operation. Then, in step S2 of FIG. 4A, the shutter 119 confirms that laser light is not emitted out of the apparatus by blocking the laser light. Next, in step S3 of FIG. 4A, laser light is generated by turning on the laser light source 101. In step S4 of FIG. 4A, the output of the laser light is measured by the output measurement unit 116.

Next, in step S5 of FIG. 4A, the irradiation amount irradiated on the fundus of the subject's eye is converted by the conversion unit 117 based on the conversion formula between the output of the laser light and the irradiation amount irradiated on the fundus of the subject's eye stored in the storage unit 117a. In other words, the amount of light enters in the subject's eye is determined.

In step S6 of FIG. 4A, the determination unit 300 determines whether the converted irradiation amount is equal to or less than a predetermined value. If it is determined that the irradiation amount is equal to or less than the predetermined value (YES in step S6), in step S7 of FIG. 4A, the control unit 400 switches the shutter 119 from the shutter closed state to shutter opened state. Then, in step S8 of FIG. 4A, the control unit 400 performs alignment using a known alignment device. In steps S9 to S11 of FIG. 4A, the control unit 400 performs processing with laser light.

If it is determined that the converted irradiation amount is greater than the predetermined value (NO in step S6), in step S12 of FIG. 4A, the control unit 400 serving as a light amount adjustment unit adjusts the light amount. In step S13, the system control unit 600 determines whether the adjustment has been succesufully performed or not by determining whether the value of the laser light amount has been changed. If it is determined that the light amount adjustment has been succesufully performed (YES in step S13), the processing returns to step S4 of FIG. 4A.

If it is determined that the light amount adjustment has not been successfully performed (NO in step S13), in step S14 of FIG. 4A, a visual warning is displayed on the display unit, or a sound warning is issued. Examples of a state in which a warning is issued include cases in which the apparatus is not acting normally or has broken down, such as when the output from the laser light source cannot be controlled or when an optical unit member is damaged and an abnormal output value is detected. If a warning is issued, the operator can reliably recognize that an abnormality has occurred.

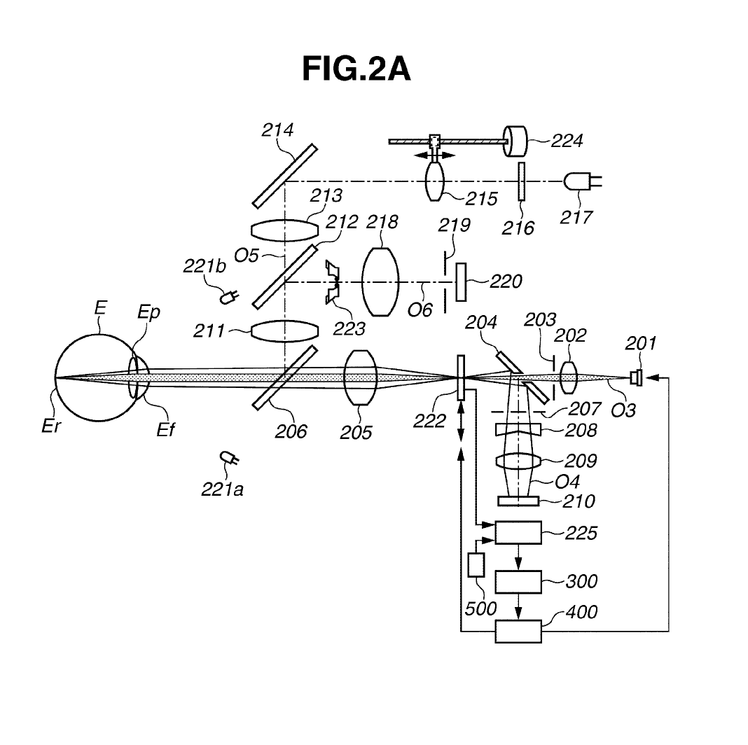

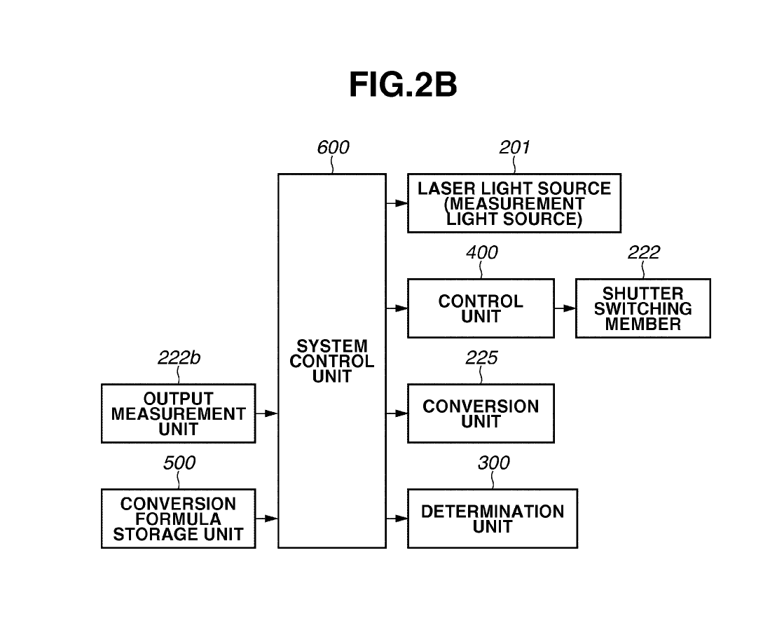

Next, a second exemplary embodiment according to the present invention will be described with reference to FIGS. 2A and 2B. FIGS. 2A and 2B illustrate an example of a schematic configuration of an eye refractive power measurement apparatus, which is an example of the ophthalmologic apparatus according to the present exemplary embodiment.

### (Fixation Target Projection Optical System and Alignment Light Receiving Optical System)

A fixation target projection optical system and an alignment light receiving optical system, which is used for both anterior eye observation and alignment detection of the subject's eye, are arranged in a reflection direction of a dichroic mirror 206. A lens 211, a dichroic mirror 212, a lens 213, a folding mirror 214, a lens 215, a fixation target 216, and a fixation target illumination light source 217 are arranged in that order on a fixation target projection optical system light path 05.

During fixation guidance, a projection light beam from the lit fixation target illumination light source 217 illuminates the fixation target 216 from the rear side, and is projected on a fundus Er of the subject's eye E via the lens 215, the folding mirror 214, the lens 213, and the dichroic mirror 212. To realize a fogging state by performing diopter guidance of the subject's eye E, the lens 215 is configured so that it can be moved in the optical axis direction by a fixation guidance motor 224.

Further, an alignment prism diaphragm 223, a lens 218, a diaphragm 219, and an image sensor 220 are arranged in that order on a light path 06 in the reflection direction of the dichroic mirror 212. Consequently, anterior eye observation and alignment detection of the subject's eye can be performed. The alignment prism diaphragm 223 is driven by an alignment prism diaphragm drive solenoid (not illustrated), and the diaphragm 219 is driven by a diaphragm drive solenoid (not illustrated). Insertion/retraction of the alignment prism diaphragm 223 allows switching between the alignment when the alignment prism diaphragm 223 is on the light path 06, and the anterior eye observation or transillumination observation when the alignment prism diaphragm 223 is retracted from the light path.

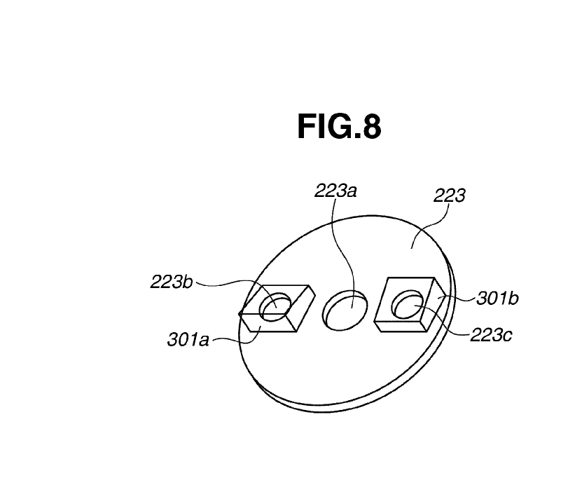

The alignment prism diaphragm 223 includes three apertures (an aperture 223a in the center portion and apertures 223b and 223c at either end in the left and right directions, respectively) on a disc-shaped diaphragm plate as illustrated in FIG. 8. Further, alignment prisms 301a and 301b that only allow light having a wavelength of around 880 nm, for example, to pass through are respectively attached to the dichroic mirror 212 side of the apertures 223b and 223c at either end in the left and right directions.

Anterior eye illumination light sources 221a and 221b, which have a wavelength of around 780 nm, for example, are arranged diagonally in front of the anterior eye section of the subject's eye E. The light beam from the subject's eye anterior eye section illuminated by the anterior eye illumination light sources 221a and 221b forms an image on a light-receiving sensor of the image sensor 220 via the dichroic mirror 206, the lens 211, the dichroic mirror 212, and the center aperture 223a of the alignment prism diaphragm 223. The center aperture 223a of the alignment prism diaphragm 223 is configured so that light having a wavelength of 780 nm or more from the anterior eye illumination light sources 221a and 221b passes through.

### (Alignment)

The light source for alignment detection is also used as a measurement light source 201 for measuring the refractive power of the eye. During alignment, a semi-transparent diffusion plate 222c is inserted in the optical path by a diffusion plate drive solenoid.

The position where the diffusion plate 222c is inserted is a primary image forming position by a projection lens 202 of the measurement light source 201, and is a focus position of a lens 205. Consequently, an image of the measurement light source 201 is temporarily formed on the diffusion plate 222c, which then serves as a secondary light source, and that image is projected from the lens 205 as a thick parallel light beam toward the subject's eye E.

This parallel light beam is reflected by the subject's eye cornea Ef and forms a bright spot image. Then, a part of the light beam is again reflected by the dichroic mirror 206. This light beam passes through the lens 211, is reflected by the dichroic mirror 212, passes through the alignment prism diaphragm 223, is converged by the lens 218, and forms an image on the image sensor 220.

In other words, the light beams split by the apertures 223a, 223b, and 223c of the alignment prism diaphragm 223 and the prisms 301a and 301b are formed as index images Ta, Tb, and Tc on the image sensor 220. Further, bright spot images 221a′ and 221b′ of the anterior eye illumination light sources 221a and 221b are captured by the image sensor 220 along with the subject's eye anterior eye section illuminated by the anterior eye illumination light sources 221a and 221b.

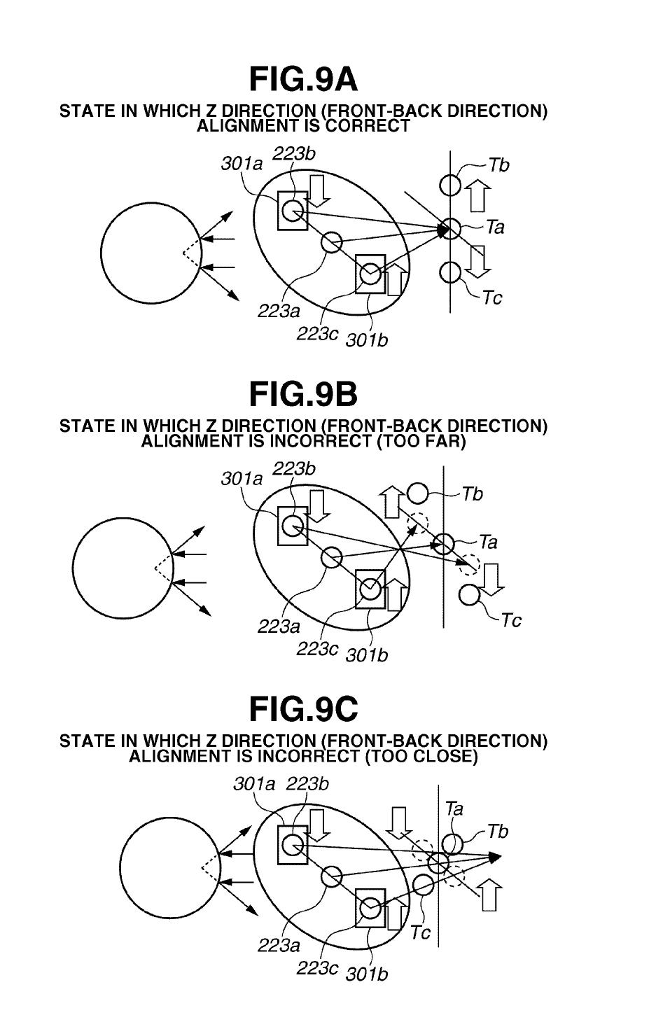

As illustrated in FIG. 9A, alignment is completed when the three cornea bright spots Ta, Tb, and Tc have been arranged in a line in the direction orthogonal to the horizontal direction. If the alignment in the Z direction (front-back direction) is too far, the alignment state looks like that illustrated in FIG. 9B, while if the alignment in the Z direction is too close, the alignment state looks like that illustrated in FIG. 9C.

### (Refractive Power Measurement)

The optical system relating to a light path 03 is for measuring the refractive power of the eye. A light beam emitted from the measurement light source 201 is formed into a primary image in front of the lens 205 by the lens 202 while being narrowed by a diaphragm 203, then passes through the lens 205 and the dichroic mirror 206, and is projected on the center of the pupil of the subject's eye E. That light beam is formed into an image at the fundus Er. The reflection light passes through the center of the pupil of the subject's eye E and is again enters into the lens 205. The incident light beam passes through the lens 205, and then is reflected by the peripheral part of a perforated mirror 204.

The reflected light beam is subjected to pupil separation by using a diaphragm 207 that is approximately conjugated with the subject's eye pupil Ep, and is projected as a ring image on the light-receiving surface of an image sensor 210. If the subject's eye E is an emmetropic eye, this ring image is a predetermined circle. If the subject's eye E is a myopic eye, the curvature of the circle decreases, while if the subject's eye E is a hypermetropic eye, the curvature of the circle increases. If the subject's eye E is astigmatic, the ring image becomes an ellipse, in which the angle formed by the horizontal axis and the long axis of the ellipse is the astigmatic axis angle. Refractive power is determined based on the coefficient of this ellipse.

### (Ophthalmologic Light Amount Adjustment Apparatus)

The measurement light source 201 for measuring the refractive power of the eye is also used as the light source for an ophthalmologic light amount adjustment apparatus. A super luminescent diode (SLD) is used for the light source 201, which is for generating laser light. Further, considering the fact that an eye is to be measured, near-infrared light is suitable for this wavelength. Here, SLD light having a wavelength of 880 nm is used as an example. The lens 202, the diaphragm 203 that is approximately conjugated with the pupil Ep of the subject's eye E, and the perforated mirror 204 are arranged in that order on the light path 03 for projecting the light beam from the SLD light source on the subject's eye E.

In addition, a shutter switching member 222 (an output measurement sensor 222b as a light blocking member and an aperture 222a as a transmissive member) that switches between blocking the projection light beam and passing the projection light beam, the lens 205, and the dichroic mirror 206 that reflects all of the visible light from the subject's eye E side and reflects a part of the laser light are arranged in that order.

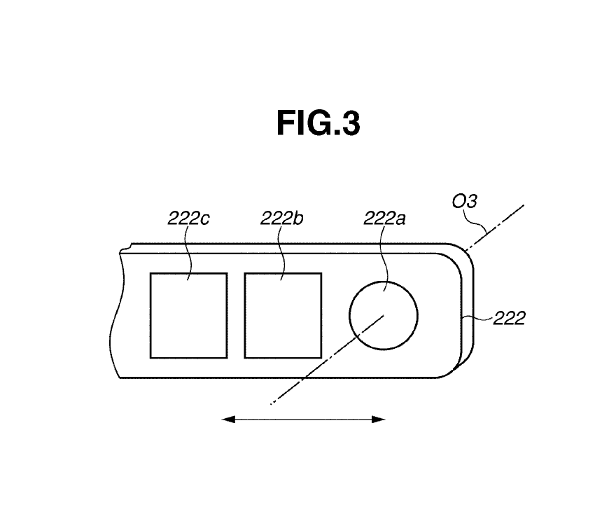

FIG. 3 illustrates the shape of the shutter switching member 222 on the light path 03 serving as a perpendicularly-displaceable blocking member switching unit. In addition to the aperture 222a, the shutter switching member 222 includes the output measurement sensor 222b that measures laser output and the diffusion plate 222c used for the alignment that is described below. When the output measurement sensor 222b is positioned on the light path 03, the shutter switching member 222 is in a light-blocking state (first state).

Further, when the aperture 222a is on the light path 03, the shutter switching member 222 is in an opened state (second state), so that the projection light beam is projected on the subject's eye. When the output measurement sensor 222b is positioned on the light path 03, the output measurement sensor 222b can detect the laser output amount.

The output measurement sensor 222b may be any unit, such as a line sensor or a two-dimensional sensor like a charge-coupled device (CCD), or a power meter, as long as it can detect light. The output measurement sensor 222b is arranged at a conjugated position with the fundus of the subject's eye, so that the laser irradiation amount irradiated on the fundus can be converted from the output of the output measurement sensor 222b in the manner described below. Further, the diaphragm 207 that is approximately conjugated with the pupil Ep and includes an annular slit, a light beam splitting prism 208, a lens 209, the image sensor 210, a conversion unit 225, the determination unit 300, and the control unit 400 are arranged in that order on a light path 04 in the reflection direction of the perforated mirror 204.

### (Irradiation Light Amount Conversion Method Performed by the Light Source Light Amount Adjustment Apparatus)

The conversion method according to the present exemplary embodiment for converting by the conversion unit 117 the output amount of the laser light source into a laser irradiation amount, such as a laser power, that is irradiated on the fundus or cornea surface of the subject's eye is as follows.

For the conversion, a conversion formula like that illustrated by the patterns in FIGS. 7A to 7D, for example, is used. More specifically, a conversion formula is used that is stored in a storage unit 500 as relation information representing the relationship between the output amount measured by the output measurement sensor 222b and the laser irradiation amount irradiated on the cornea or fundus of the subject's eye via the optical system (members 202, 205, and 206). This relation information is determined based on the rate of attenuation in the light amount on the light path from the light source to the subject's eye and the rate of attenuation in the light amount at the subject's eye.

By using this conversion formula, the actual laser irradiation amount on the subject's eye with respect to the laser output measurement value can be determined as a conversion value based on the rate of attenuation due to the membrane properties and the transmissivity of the optical members configuring the projection optical system, or based on experimentally-determined rate of attenuation data.

When experimentally determining this conversion formula, a sensor such as a power meter is arranged at the cornea or fundus position of the subject's eye E, and the shutter switching member 222 is switched to the aperture 222a state (shutter open). The measurement value at that time is taken as y. Further, if the measurement value of the output measurement sensor when the shutter switching member 222 is switched to the output measurement sensor 222b state (shutter closed) is taken as x, then a relational expression between y and x can be approximately derived. The conversion is performed by inputting that relational expression into the conversion unit 225 as a conversion table.

If the measurement mode for measuring eye information about the subject's eye is switched by changing the configuration of the optical members in the projection system, the conversion formula may be changed based on the measurement mode.

### (Laser Irradiation Determination)

The determination of laser irradiation on the subject's eye according to the present exemplary embodiment is performed as follows. The shutter switching member 222 is inserted and the output measurement sensor 222b is arranged on the light path 03. In this state, the conversion unit 225 converts the laser irradiation amount to be irradiated on the fundus of the subject's eye based on an output from the output measurement sensor 222b and a conversion formula stored in the storage unit 500 (i.e., relation information representing the relationship between the laser output amount and the laser irradiation amount).

If the conversion result is within a permissible range (less than a predetermined value), the shutter switching member 222 is operated and the light path 03 is opened. Alternatively, the diffusion plate 222c is arranged. Further, if the conversion result is not within the permissible range (equal to or greater than the predetermined value), the control unit 400 controls, for example, the current or the voltage of the laser light source to reduce the laser light amount.

Specifically, the determination unit 300 determines whether the irradiation light amount is within the permissible range based on an output from the conversion unit 225. Then, if it is determined by the determination unit 300 that the irradiation light amount is within the permissible range, the control unit 400 executes switching to the second state (shutter open) of the shutter switching member 222. On the other hand, if it is determined by the determination unit 300 that the irradiation light amount is not within the permissible range, the control unit 400 maintains the first state (shutter closed) of the shutter switching member 222.

Further, the control unit 400 reduces the output of the light source 201 and repeats the measurement by the output measurement sensor 222b, the conversion by the conversion unit 225, and the determination by the determination unit 300 until it is determined by the determination unit 300 that the irradiation light amount is within the permissible range. Thus, in the present exemplary embodiment, the output measurement sensor 222b, the conversion unit 225, the determination unit 300, and the control unit 400 are the constituent elements of the light source light amount adjustment apparatus. The determination unit 300 can also be incorporated in the control unit 400.

### (Flowchart of the Overall Apparatus)

The above configuration will now be described with reference to the block diagram illustrated in FIG. 2B and the flowchart illustrated in FIG. 4B. The system control unit 600 illustrated in FIG. 2B performs overall control of the constituent elements, such as the laser light source 201, the control unit 400, the conversion unit 225, and the determination unit 300.

In step S1′ of FIG. 4B, measurement starts. Then, in step S2 of FIG. 4B, the control unit 400 confirms that laser light is not emitted out of the apparatus by blocking the laser light. Next, in step S3 of FIG. 4B, laser light is generated by turning on the laser light source 201 (also works as a measurement light source). In step S4 of FIG. 4B, the output of the laser light is measured by the output measurement unit 222b.

Next, in step S5 of FIG. 4B, the irradiation amount to be irradiated on the fundus of the subject's eye is converted by the conversion unit 225 based on the laser output and the conversion formula between the output of the laser light stored in the storage unit 500 and the irradiation amount irradiated on the fundus of the subject's eye. In other words, the amount of light enters in the subject's eye is determined.

In step S6 of FIG. 4B, the determination unit 300 determines whether the converted irradiation amount is equal to or less than a predetermined value. If it is determined that the converted irradiation amount is equal to or less than the predetermined value (YES in step S6), in step S7′ of FIG. 4B, the control unit 400 switches the shutter switching member 222 from shutter closed state to shutter opened state. Then, in step S8′ of FIG. 4B, the control unit 400 performs the above-described auto-alignment. In steps S9′ to S11′ of FIG. 4B, the control unit 400 performs measurement based on laser light.

If it is determined that the converted irradiation amount is greater than the predetermined value (NO in step S6), in step S12 of FIG. 4B, the control unit 400 serving as a light amount adjustment unit adjusts the light amount of the laser light. In step S13, the system control unit 600 determines whether the adjustment has been succesufully performed or not by determining whether the value of the laser light amount has changed. If it is determined that the light amount adjustment has been succesufully performed (YES in step S13), the processing returns to step S4 of FIG. 4B.

If it is determined that the light amount adjustment has not been succesufully performed (NO in step S13), in step S14 of FIG. 4B, a visual warning is displayed on the display unit, or a sound warning is issued. Examples of a state in which a warning is issued include cases in which the apparatus is not acting normally or has broken down, such as when the output from the laser light source cannot be controlled or when an optical unit member is damaged and an abnormal output value is detected. If a warning is issued, the operator can reliably recognize that an abnormality has occurred.

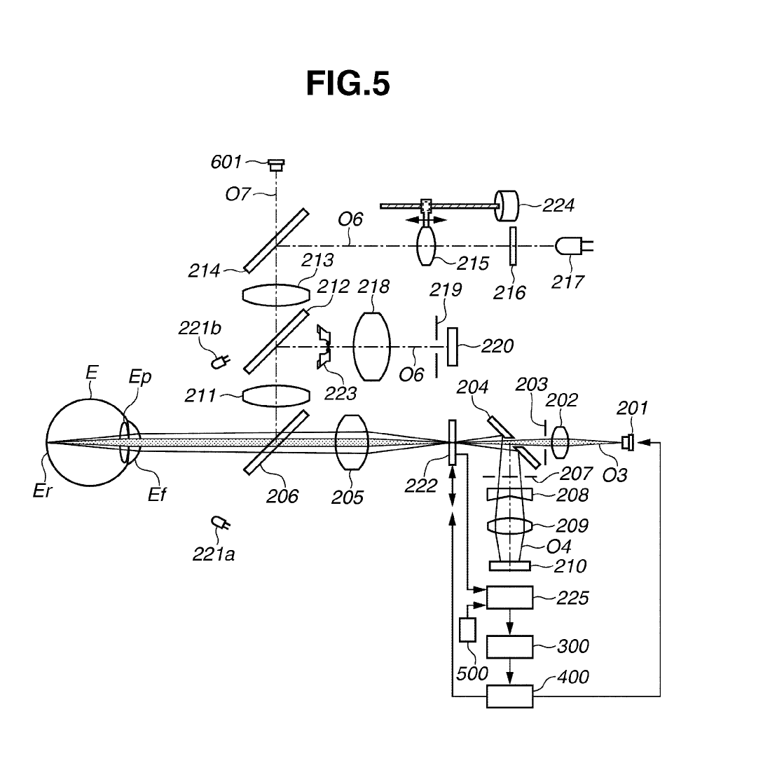

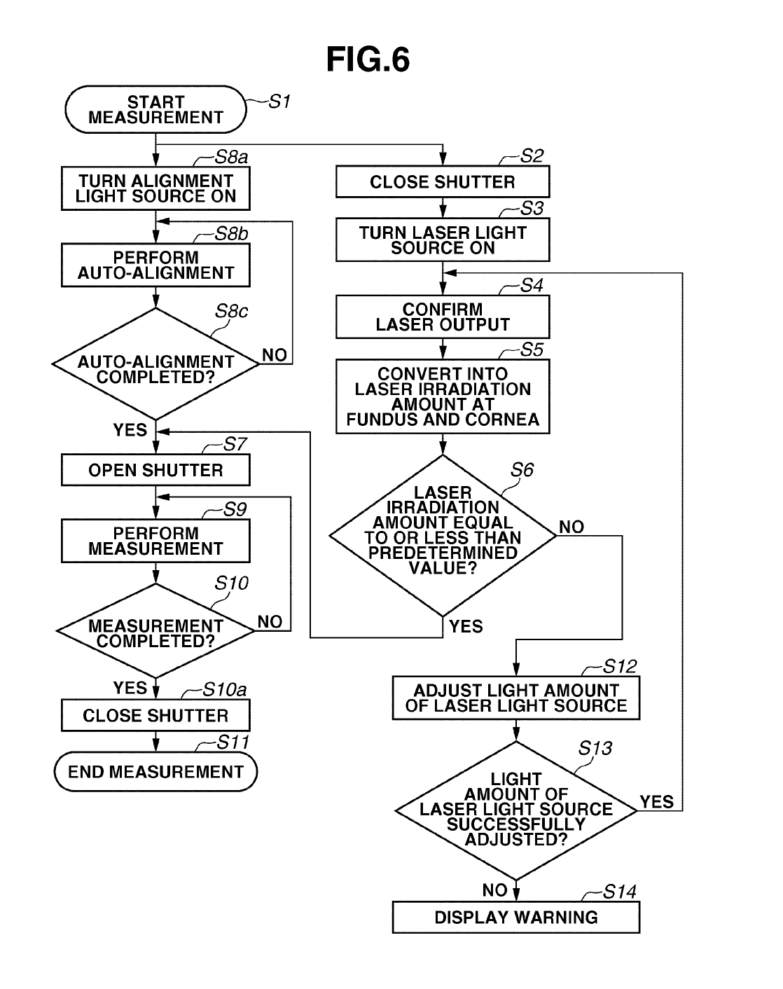

An ocular refractive power measurement apparatus according to a third exemplary embodiment will now be described with reference to FIGS. 5 and 6. This ocular refractive power measurement apparatus differs from the second exemplary embodiment in that a low-power alignment light source 601 that emits infrared light rather than visible light, and does not have a harmful effect on a test subject, is arranged as a separate light source on a light path 07. Further, this alignment light source 601 can be arranged at a similar position to the anterior eye illumination light sources 221a and 221b in place of the anterior eye illumination light sources 221a and 221b.

In this case, the alignment performed in the second exemplary embodiment in step S8 of FIG. 4 can be replaced by steps S8a, S8b, and S8c of FIG. 6. More specifically, in the second exemplary embodiment, steps to determine laser light beam irradiation are performed in steps S2 to S6, S12, and S13 of FIG. 4, then an alignment step is performed in step S8 of FIG. 4, and then the measurement step is performed in step S9 of FIG. 4. On the other hand, in the present exemplary embodiment, the steps to determine laser light beam irradiation in steps S2 to S6, S12, and S13 of FIG. 6 can be performed simultaneously with the alignment step in steps S8a, S8b, and S8c of FIG. 6. Consequently, the time until measurement is started can be shortened.

### Modified Example 1

In the above exemplary embodiments, although the present invention is applied in an ocular refractive power measurement apparatus, the present invention can also be applied to an optical coherence tomography (OCT) apparatus that utilizes a laser source for generating laser light, a fundus camera, or even to a non-ophthalmologic OCT apparatus.

### Modified Example 2

The calculation formula stored in the storage unit can be replaced with a calculation method that stores only discrete data and calculates the values between stored data based on a known interpolation method.

### Modified Example 3

In the above exemplary embodiments, the shutter 119 or shutter switching member 222 arranged on the light path switches the blocked state and the opened state. However, the present invention is not limited to this. The switching between the blocked state and the opened state may also be performed by replacing the blocked state with a light-restricted state (state in which, for example, the light amount that passes through is restricted to 1/10 of the incident light amount using an ND filter or the like as a restriction member).

### Other Exemplary Embodiments

Further, as an ophthalmologic control method for adjusting the light amount when irradiating a predetermined site of a subject's eye with a light beam emitted from a light source via the optical system, the present invention may further include a step for measuring the light amount emitted from the light source with a measurement unit, and a step for controlling the light amount emitted from the light source based on relation information representing a relationship between the light amount emitted from the light source and the light amount irradiated on the subject's eye, and the light amount measured by the measurement unit.

Further, an ophthalmologic control method can be realized by supplying software (a program) for realizing the functions of the above-described exemplary embodiments to a system or an apparatus via a network or via various storage media, and by causing a computer (a central processing unit (CPU) or a micro processing unit (MPU)) of the system or apparatus to read and execute the program.

According to the present invention, subject's eye irradiation can be performed at an appropriate light amount by controlling the light amount emitted from a light source based on relation information representing a relationship between a light amount emitted from the light source and the light amount irradiated on a subject's eye, and the light amount emitted from the light source and measured by a measurement unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-234648 filed Oct. 24, 2012, which is hereby incorporated by reference herein in its entirety.

What is claimed is:

1. An ophthalmologic apparatus comprising:
a measurement unit configured to measure a light amount emitted from a light source; and
a control unit configured to control a light amount emitted from the light source based on relation information representing a relationship between a light amount emitted from the light source and a light amount irradiated on a subject's eye, and the light amount measured by the measurement unit.

2. The ophthalmologic apparatus according to claim 1, further comprising a switching unit configured to switch between a first state in which a restriction member for restricting incidence of light from the light source on the subject's eye is positioned on a light path between the light source and the subject's eye, and a second state in which the restriction member is retracted from the light path,
wherein the measurement unit is configured to measure a light amount emitted from the light source in the first state.

3. The ophthalmologic apparatus according to claim 2, further comprising:
a first determination unit configured to determine a light amount entering in the subject's eye based on the relation information and the light amount measured by the measurement unit; and
a second determination unit configured to determine whether the light amount determined by the first determination unit is equal to or less than a predetermined value,
wherein the control unit is configured to reduce the light amount emitted from the light source if it is determined by the second determination unit that the light amount determined by the first determination unit is greater than the predetermined value.

4. The ophthalmologic apparatus according to claim 3, wherein the control unit is configured to reduce the light amount emitted from the light source until it is determined that the light amount determined by the first determination unit is equal to or less than the predetermined value.

5. The ophthalmologic apparatus according to claim 3, wherein the switching unit is configured to switch from the first state to the second state if it is determined by the second determination unit that the light amount determined by the first determination unit is equal to or less than the predetermined value.

6. The ophthalmologic apparatus according to claim 1, further comprising a splitting unit configured to split the light emitted from the light source on a light path from the light source to the measurement unit,
wherein the measurement unit is configured to measure an amount of light different from light that is heading toward the subject's eye among the light split by the splitting unit.

7. The ophthalmologic apparatus according to claim 1,
wherein the measurement unit is provided on a restriction member configured to restrict incidence of light from the light source on the subject's eye, and
wherein the measurement unit is arranged on a light path between the light source and the subject's eye.

8. The ophthalmologic apparatus according to claim 7, wherein the restriction member includes a transmissive member, and the transmissive member is arranged on the light path in a state in which the measurement unit is retracted from the light path.

9. The ophthalmologic apparatus according to claim 1, wherein the relation information is determined based on an amount of attenuation in the light amount from the light source to the subject's eye.

10. The ophthalmologic apparatus according to claim 1, wherein the relation information is determined based on an amount of attenuation in the light amount on a light path from the light source to the subject's eye and an amount of attenuation in the light amount in the subject's eye.

11. The ophthalmologic apparatus according to claim 3, further comprising a warning unit configured to issue a warning if it is determined by the second determination unit that the light amount determined by the first determination unit is greater than a predetermined value.

12. An ophthalmologic control method comprising:
measuring with a measurement unit a light amount emitted from a light source; and
controlling a light amount emitted from the light source based on relation information representing a relationship between a light amount emitted from the light source and a light amount irradiated on a subject's eye, and the light amount measured by the measurement unit.

13. A non-transitory computer-readable recording medium on which a program is recorded that that causes a computer to execute the method according to claim 12.
